(19) United States
(12) Patent Application Publication
(10) Pub. No.: US 2023/0172508 A1
(43) Pub. Date: Jun. 8, 2023

(54) COGNITIVE NOTIFICATIONS CHOREOGRAPHER

(71) Applicant: INTERNATIONAL BUSINESS MACHINES CORPORATION, ARMONK, NY (US)

(72) Inventors: Hernan A. Cunico, Holly Springs, NC (US); Craig M. Trim, Ventura, CA (US); John M. Ganci, JR., Raleigh, NC (US); Martin G. Keen, Cary, NC (US)

(21) Appl. No.: 17/457,270

(22) Filed: Dec. 2, 2021

(51) Int. Cl. A61B 5/16; G06N 3/08; G10L 15/26; G10L 25/63; G10L 25/66; G06V 40/20

(52) U.S. Cl. CPC A61B 5/165; G06N 3/08; G10L 15/26; G10L 25/63; G10L 25/66; G06V 40/23

(57) ABSTRACT

The present invention may include a computer receives one or more communications and data from a sensor. The computer determines an activity of a user based on the received data, wherein the activity is determined by a trained neural network. The computer calculates a stress coefficient based on the activity and the data. The computer generates a cognitive notifications choreographer model from the stress coefficient and one or more communications and causes a communication software to reply to the one or more communications based on the cognitive notifications choreographer model determining the stress coefficient above a threshold value.

### BACKGROUND

The present invention relates, generally, to the field of computing, and more particularly to machine learning.

Machine learning (ML) is the study of computer algorithms that improve automatically through experience and by the use of data. Machine learning is seen as a part of artificial intelligence (AI). Machine learning algorithms build a model based on sample data, known as training data or a training set, in order to make predictions or decisions without being explicitly programmed to do so. Machine learning algorithms are used in a wide variety of applications, such as medicine, computer vision, and natural language processing, where it is difficult or unfeasible to develop conventional algorithms to perform the needed tasks. A subset of machine learning is closely related to computational statistics, which focuses on making predictions using computers. The study of mathematical optimization delivers methods, theory and application domains to the field of machine learning. Data mining is a related field of study, focusing on exploratory data analysis through unsupervised learning. In its application across business problems, machine learning is also referred to as predictive analytics.

### SUMMARY

According to one embodiment, a method, computer system, and computer program product for cognitive notification choreography is provided. The present invention may include a computer receives one or more communications and data from a sensor. The computer determines an activity of a user based on the received data, wherein the activity is determined by a trained neural network. The computer calculates a stress coefficient based on the activity and the data. The computer generates a cognitive notifications choreographer model from the stress coefficient and one or more communications and causes a communication software to reply to the one or more communications based on the cognitive notifications choreographer model determining the stress coefficient above a threshold value.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment;

FIG. 2 is an operational flowchart illustrating a cognitive notifications choreographer process according to at least one embodiment;

FIG. 3 is a block diagram of internal and external components of computers and servers depicted in FIG. 1 according to at least one embodiment;

FIG. 4 depicts a cloud computing environment according to an embodiment of the present invention; and

FIG. 5 depicts abstraction model layers according to an embodiment of the present invention.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Embodiments of the present invention relate to the field of computing, and more particularly to utilizing machine learning (ML) for determination of cognitive performance and dynamic remediation of stress level of a user. The following described exemplary embodiments provide a system, method, and program product to, among other things, determine a stress level of a user and take stress relieving actions by controlling the incoming communications. Therefore, the present embodiment has the capacity to improve the technical field of machine learning and user interface by calculating a stress coefficient of a user and using the stress coefficient to monitor and automatically control communications.

As previously described, ML is the study of computer algorithms that improve automatically through experience and by the use of data. Machine learning is seen as a part of artificial intelligence (AI). Machine learning algorithms build a model based on sample data, known as training data or a training set, in order to make predictions or decisions without being explicitly programmed to do so. Machine learning algorithms are used in a wide variety of applications, such as medicine, computer vision, and natural language processing where it is difficult or unfeasible to develop conventional algorithms to perform the needed tasks. A subset of machine learning is closely related to computational statistics, which focuses on making predictions using computers. The study of mathematical optimization delivers methods, theory and application domains to the field of machine learning. Data mining is a related field of study, focusing on exploratory data analysis through unsupervised learning. In its application across business problems, machine learning is also referred to as predictive analytics.

The more collaboration tools are used, the more users get dragged into a multitude of conversations. Each discourse is typically made up of a plurality of individual messages, where each message may trigger a separate notification (such as a pop-up message) that may become overwhelmingly distracting and directly impact the user performance and productivity.

Collaborative messaging is not the sole source of interruptions. Email notifications, calendar alerts, text messages, and spam calls each add to the user disruptions. Typically, there is a direct relationship between the focus needed to complete a task, the importance of that task, the interruptions to the stress level of a user.

Frequently, depending on the tools used, a user has a few options to control the flow of those notifications. The user may mute a channel, a conversation, or allow notifications for direct messages only. By the same measure, a user may set his phone to a vibrate mode or do not disturb (DND) mode and all the calls may be forwarded to a voice mail. None of these methods are efficient given that they are very static.

Therefore, computerized communications, such as messages, emails, chats and other collaborative environments, require a user to engage in a multitude of conversations. In addition, each collaborative environment typically includes a plurality of individual messages when each message may trigger a separate notification that is overwhelmingly distracting and increasing a stress level of the user, thus directly impacting the performance and productivity of the user. As such, it may be advantageous to, among other things, implement a system that analyzes stress levels of the user by utilizing machine learning of data received from sensors, such as a microphone, camera or other user inputs, and, based on the analysis, determine whether an active communication should be displayed, delayed or answered by an AI service.

According to one embodiment, a cognitive notifications choreographer program may analyze data from various sensors and user inputs to dynamically determine a cognitive notifications choreographer model that evaluates a stress factor and relevancy of the communication to the task and based on the factors takes stress relieving actions to increase productivity and reduce stress of the user. The cognitive notifications choreographer program may contextually associate activities with communications, notifications and user stress level to derive a Relative Stress Coefficient (RSC). Furthermore, the cognitive notifications choreographer program may use a discrete method for contextually determining handling of interruptions through application of a Cognitive Notifications Choreographer Model (CNCM).

Moreover, the cognitive notifications choreographer program may selectively automate actions to reduce stress of the user through application of a CNCM, that may control chat bots, utilize AI-driven tasks, and display prioritized visual representation of communications.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method, and program product to create a notifications choreographer model that identifies user stress levels and reassigns, delays, or displays the communications dynamically based on the stress level of the user and relevancy of the incoming communication to the current task of the user.

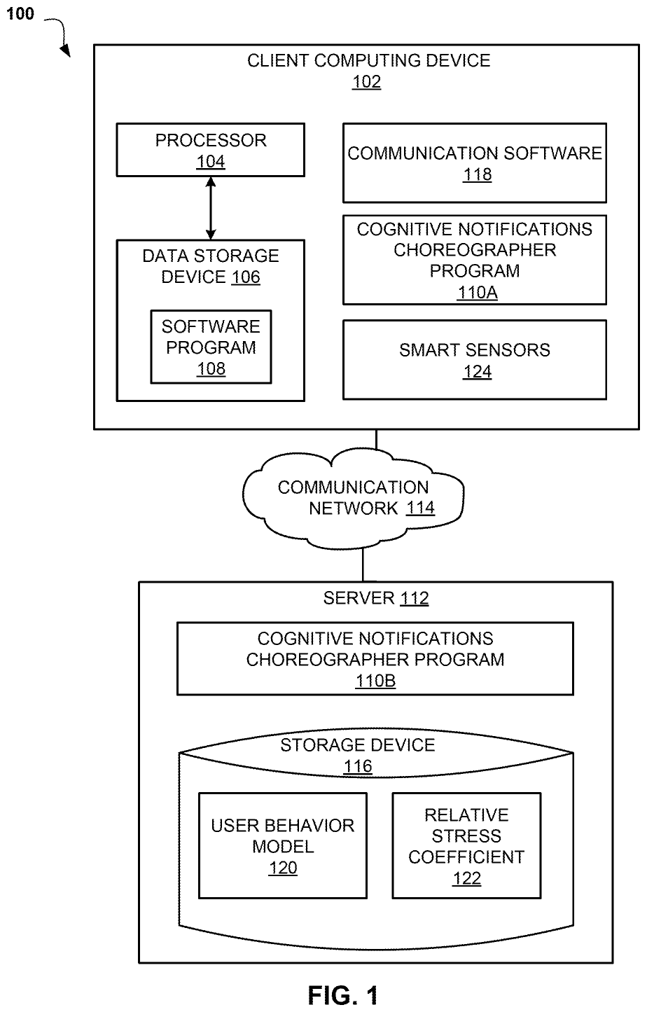

Referring to FIG. 1, an exemplary networked computer environment 100 is depicted, according to at least one embodiment. The networked computer environment 100 may include client computing device 102 and a server 112 interconnected via a communication network 114. According to at least one implementation, the networked computer environment 100 may include a plurality of client computing devices 102 and servers 112, of which only one of each is shown for illustrative brevity.

The communication network 114 may include various types of communication networks, such as a wide area network (WAN), local area network (LAN), a telecommunication network, a wireless network, a public switched network and/or a satellite network. The communication network 114 may include connections, such as wire, wireless communication links, or fiber optic cables. It may be appreciated that FIG. 1 provides only an illustration of one implementation and does not imply any limitations with regard to the environments in which different embodiments may be implemented. Many modifications to the depicted environments may be made based on design and implementation requirements.

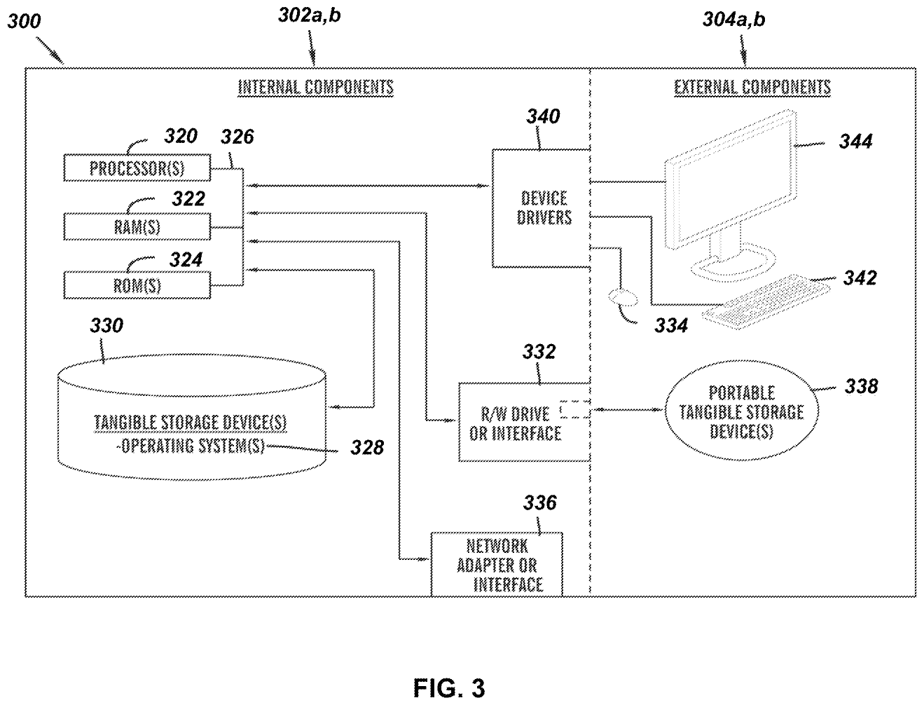

Client computing device 102 may include a processor 104, smart sensors 124 and a data storage device 106 that is enabled to host and run a software program 108, a communication software 118 and a cognitive notifications choreographer program 110A and communicate with the server 112 via the communication network 114, in accordance with one embodiment of the invention. Client computing device 102 may be, for example, a mobile device, a telephone, a personal digital assistant, a netbook, a laptop computer, a tablet computer, a desktop computer, or any type of computing device capable of running a program and accessing a network. Communication software 118 may be any software that enables communications between users such as a chat, a videoconference, or a voice call. Smart sensors 124 may be sensors or devices that may be used to determine a stress level of a user. For example smart sensors 124 may be a microphone, a camera, a touchscreen, a keyboard, a global positioning sensor or other biometrical device that may be utilized to determine the stress level of the user. As will be discussed with reference to FIG. 3, the client computing device 102 may include internal components 302a and external components 304a, respectively.

The server computer 112 may be a laptop computer, netbook computer, personal computer (PC), a desktop computer, or any programmable electronic device or any network of programmable electronic devices capable of hosting and running a cognitive notifications choreographer program 110B and a storage device 116 storing user behavior model 120 records and relative stress coefficient 122 records and communicating with the client computing device 102 via the communication network 114, in accordance with embodiments of the invention. As will be discussed with reference to FIG. 3, the server computer 112 may include internal components 302b and external components 304b, respectively. The server 112 may also operate in a cloud computing service model, such as Software as a Service (SaaS), Platform as a Service (PaaS), or Infrastructure as a Service (IaaS). The server 112 may also be located in a cloud computing deployment model, such as a private cloud, community cloud, public cloud, or hybrid cloud.

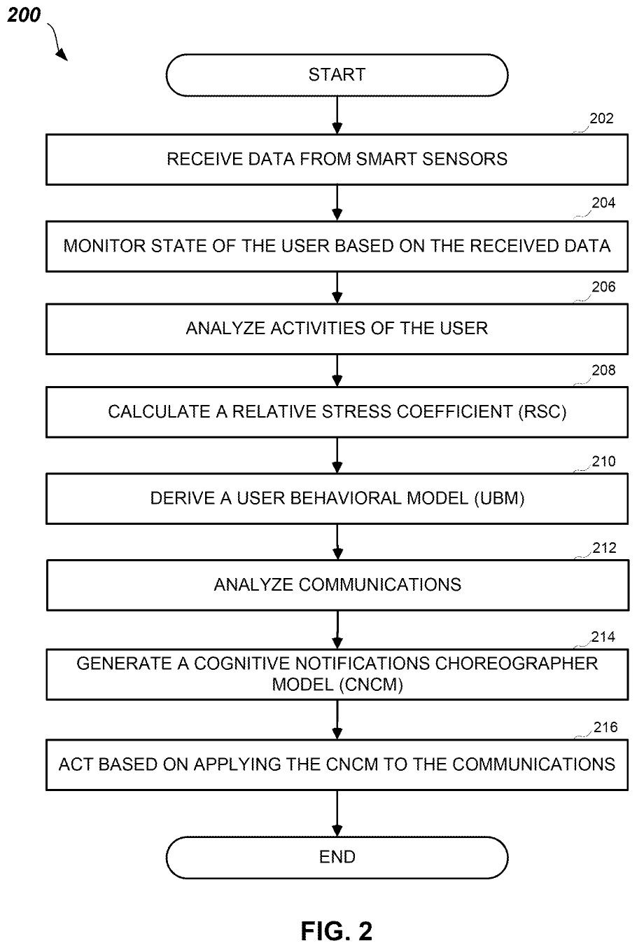

According to the present embodiment, the cognitive notifications choreographer (CNC) program 110A, 110B may be a program capable of monitoring speech, text, and body movements of the user in order to predict user stress level and controlling communications using machine learning model to reduce the stress level of the user and thus increase productivity of communications. By controlling, the machine learning model may control the flow of communications, act on behalf of the user such as answer to communications and display to the user the actions taken by the program. The cognitive notifications choreographer method is explained in further detail below with respect to FIG. 2.

Referring now to FIG. 2, an operational flowchart illustrating a cognitive notifications choreographer process 200 is depicted, according to at least one embodiment. At 202, the cognitive notifications choreographer (CNC) program 110A, 110B receives data from smart sensors. According to an example embodiment, the CNC program 110A, 110B may request a user, via a graphical user interface (GUI) to opt in for the services that allows CNC program 110A, 110B to gather user-related information, such as speech voice and body movement data received from smart sensors 124 or other sensors that are related to the user such as smart cameras, smart watches or other devices connected to communication network 114. In addition, CNC program 110A, 110B may receive all of the communications from communication software 118 for analysis during runtime.

Next, at 204, the CNC program 110A, 110B monitors a state of the user based on the received data. According to an example embodiment, the CNC program 110A, 110B may perform sentiment analysis of the monitored data utilizing trained neural networks that identify sentiment of the user from the data typed by the user or received via smart sensors 124, such as a camera and a microphone. According to an example embodiment, the CNC program 110A, 110B may identify the monitored data as a speech, text and body movements in order to identify the state and activity of the user. In another embodiment, the CNC program 110A, 110B may convert speech to text for identification of a sentiment and a topic using speech-to-text algorithms that directly correlate to the state of the user. For example, if the speech to text reveals that the user is joking that is directly related that the user is in a sate of relaxation and not under stress of workload.

Then, at 206, the CNC program 110A, 110B analyzes activities of the user. According to an example embodiment, the CNC program 110A, 110B may utilize trained neural networks or statistical models to derive data of each activity such as a type of the activity, a relative complexity (c) of the activity, an importance of a task (i), a topic of the activity, a category of the activity, a sentiment coefficient (s) and a participation coefficient (p) of the activity. For example, the trained neural network, such as IBM Watson® (IBM Watson and all IBM Watson-based trademarks and logos are trademarks or registered trademarks of International Business Machines Corporation and/or its affiliates), may receive user verbal, textual and body movement inputs and derive the activity data and relative complexity by utilizing corpus linguist methods and clustering of the data using a Gaussian mixture model (GMM). In another embodiment, the CNC program 110A, 110B may store, upload or update user data to the user behavior model 120. In another embodiment, the CNC program 110A, 110B may analyze communications and determine whether each of the communications is associated to the current activity or task of the user utilizing topic modelling and corpus linguistic techniques. In another embodiment, the CNC program 110A, 110B may analyze activities of the user based on context analysis of the monitored data, such as by using the Barnard's or Fisher's test to determine the dependencies or independencies between activities.

Next, at 208, the CNC program 110A, 110B calculates a relative stress coefficient (RSC) from the derived coefficients of the analyzed activities. According to an example embodiment, the CNC program 110A, 110B may calculate the RSC as a function of the relative complexity (c), the importance of a task (i), the sentiment coefficient (s) and the participation coefficient (p) derived from the activity data. For example, the RSC may be calculated as RSC=w₁(c)+w₂(i)+w₃(s)+w₄(p) where w₁-w₄ are weights that may be adjusted by the CNC program 110A, 110B based on the feedback received from the user or uploaded from relative stress coefficient 122 records. In another embodiment, the CNC program 110A, 110B may determine and add to the calculations of the RSC a notification that impacting the sentiment (s) to the calculations of the RSC value.

Then, at 210, the CNC program 110A, 110B derives a user behavioral model (UBM). According to an example embodiment, the CNC program 110A, 110B may derive the UBM from previous user responses and actions where the calculated RSC differentiates between different patterns of stress. These patterns may be associated with tailored intervention by the CNC program 110A, 110B to reduce the strain of stress. According to an example embodiment, the CNC program 110A, 110B may store and update the UBM in the user behavior model 120 record. For example, the CNC program 110A, 110B may calculate the RSC while the user uses various applications or activities. In addition, the CNC program 110A, 110B may display accompanying questions for the user via a GUI, and update the UBM that is used to receive sentiment data and the RSC as an input and outputs predictions comprising, a value representing how busy the user is, participation level percentage, stress indicator value, and relevance index (r) value.

Next, at 212, the CNC program 110A, 110B analyzes communications. According to an example embodiment, the CNC program 110A, 110B may analyze incoming communications for their relevancy to the activity and context using topic modelling or corpus linguistic approaches that may associate a relative relevance index (r) to each communication.

Then, at 214, the CNC program 110A, 110B generates a cognitive notifications choreographer model (CNCM). According to an example embodiment, the CNC program 110A, 110B may generate a multi-dimensional cognitive notifications choreographer model (CNCM) that correlates conversations or messages against the user activities within context and the relative stress indicator values of the UBM of the user. For example, the CNCM may be a trained neural network that is initially trained by the feedbacks of the user until it provides acceptable for the user responses. In another embodiment, the CNC program 110A, 110B may rank the conversations proportionally to the previously calculated relevance index (r) along with the relative stress indicator (s) that is associated with the current activity. In another embodiment, the CNCM model may rank communications with higher relevance index (r) and higher relative stress indicator (sᵢ) higher than other communications with less contextual significance.

Next, at 216, the CNC program 110A, 110B acts based on applying the CNCM to the communications. According to an example embodiment, the CNC program 110A, 110B may, using the CNMC model, predict what communications are more disruptive to the user based on one or more of changes of the stress coefficient, stress indicator (s), relevance index (r) and higher relative stress indicator (sᵢ). For example, the CNC program 110A, 110B may use predetermined stress indicator (s) ranges that are associated to specific remediation actions. For example, the stress coefficient values may be below a threshold value associated with low stress indicator (s) values, thus the CNC program 110A, 110B may not intervene and enable the communications to pass through to the user. In another group, the CNC program 110A, 110B may use a personalized chat bot with knowledge learned from personalized CNCM model to reply automatically (with specific confidence level) to a predetermined communication type. This remediation by the CNC program 110A, 110B may reduce the number of interruptions and filter out the set of questions that the user absolutely needs to address at various priority levels. In other embodiments, the CNC program 110A, 110B may send an email summarizing the various questions and answers responded by the personalized chat bot. In further embodiments, the CNC program 110A, 110B may discern whether a request can be addressed automatically or if the user involvement is required. For example, certain requests or communications may be fulfilled by an AI-driven system such as an autonomous vehicle AI system. For example, if a communication is a reminder to go pick up the shirts from the cleaner. Given the characteristics of the request and current context, the CNC program 110A, 110B could generate a command for the autonomous vehicle to pick up the clothes from the cleaner. Alternatively, the CNC program 110A, 110B may transfer the request to an Uber driver that may fulfill such a request. In further embodiments, the CNC program 110A, 110B may visually organize the categorization of communications using GUI. For example, the CNC program 110A, 110B may display a dashboard that summarizes all of the communications being handled by the system and enable the user to intervene in their categorizations and responses in order to train or update the CNCM model. In further embodiments, the CNC program 110A, 110B may utilize the CNCM model to discern whether a request in the communication may be addressed automatically or if the user involvement is required. Certain questions or requests in the communication may be fulfilled by an AI-driven robot such as an autonomous vehicle. For example, a user may get a reminder to go to pick up the shirts from the cleaner. The user might be too busy for that notification and incapable of finding time to do that pick up. Given the characteristics of the request and current context, the CNC program 110A, 110B may send a request to an autonomous vehicle to pick up clothes from the cleaner. Alternatively, a driver or Uber could receive a message from CNC program 110A, 110B that requests to fulfill the task.

It may be appreciated that FIG. 2 provides only an illustration of one implementation and does not imply any limitations with regard to how different embodiments may be implemented. Many modifications to the depicted environments may be made based on design and implementation requirements. For example, the CNC program 110A, 110B may take various remediation methods to reduce or maintain stress indicator (sᵢ) such as bundling communications, delaying communications before displaying to the user, or forwarding the communications to alternative users.

FIG. 3 is a block diagram 300 of internal and external components of the client computing device 102 and the server 112 depicted in FIG. 1 in accordance with an embodiment of the present invention. It should be appreciated that FIG. 3 provides only an illustration of one implementation and does not imply any limitations with regard to the environments in which different embodiments may be implemented. Many modifications to the depicted environments may be made based on design and implementation requirements.

The data processing system 302, 304 is representative of any electronic device capable of executing machine-readable program instructions. The data processing system 302, 304 may be representative of a smart phone, a computer system, PDA, or other electronic devices. Examples of computing systems, environments, and/or configurations that may represented by the data processing system 302, 304 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

The client computing device 102 and the server 112 may include respective sets of internal components 302 a,b and external components 304 a,b illustrated in FIG. 3. Each of the sets of internal components 302 include one or more processors 320, one or more computer-readable RAMs 322, and one or more computer-readable ROMs 324 on one or more buses 326, and one or more operating systems 328 and one or more computer-readable tangible storage devices 330. The one or more operating systems 328, the software program 108 and the CNC program 110A in the client computing device 102, and the CNC program 110B in the server 112 are stored on one or more of the respective computer-readable tangible storage devices 330 for execution by one or more of the respective processors 320 via one or more of the respective RAMs 322 (which typically include cache memory). In the embodiment illustrated in FIG. 3, each of the computer-readable tangible storage devices 330 is a magnetic disk storage device of an internal hard drive. Alternatively, each of the computer-readable tangible storage devices 330 is a semiconductor storage device such as ROM 324, EPROM, flash memory or any other computer-readable tangible storage device that can store a computer program and digital information.

Each set of internal components 302 a,b also includes a R/W drive or interface 332 to read from and write to one or more portable computer-readable tangible storage devices 338 such as a CD-ROM, DVD, memory stick, magnetic tape, magnetic disk, optical disk or semiconductor storage device. A software program, such as the notifications choreographer program 110A, 110B, can be stored on one or more of the respective portable computer-readable tangible storage devices 338, read via the respective R/W drive or interface 332, and loaded into the respective hard drive 330.

Each set of internal components 302 a,b also includes network adapters or interfaces 336 such as a TCP/IP adapter cards, wireless Wi-Fi interface cards, 3G, 4G or 5G wireless interface cards or other wired or wireless communication links. The software program 108 and the CNC program 110A in the client computing device 102 and the CNC program 110B in the server 112 can be downloaded to the client computing device 102 and the server 112 from an external computer via a network (for example, the Internet, a local area network or other, wide area network) and respective network adapters or interfaces 336. From the network adapters or interfaces 336, the software program 108 and the CNC program 110A in the client computing device 102 and the CNC program 110B in the server 112 are loaded into the respective hard drive 330. The network may comprise copper wires, optical fibers, wireless transmission, routers, firewalls, switches, gateway computers and/or edge servers.

Each of the sets of external components 304 a,b can include a computer display monitor 344, a keyboard 342, and a computer mouse 334. External components 304 a,b can also include touch screens, virtual keyboards, touch pads, pointing devices, and other human interface devices. Each of the sets of internal components 302 a,b also includes device drivers 340 to interface to computer display monitor 344, keyboard 342, and computer mouse 334. The device drivers 340, R/W drive or interface 332, and network adapter or interface 336 comprise hardware and software (stored in storage device 330 and/or ROM 324).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based email). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

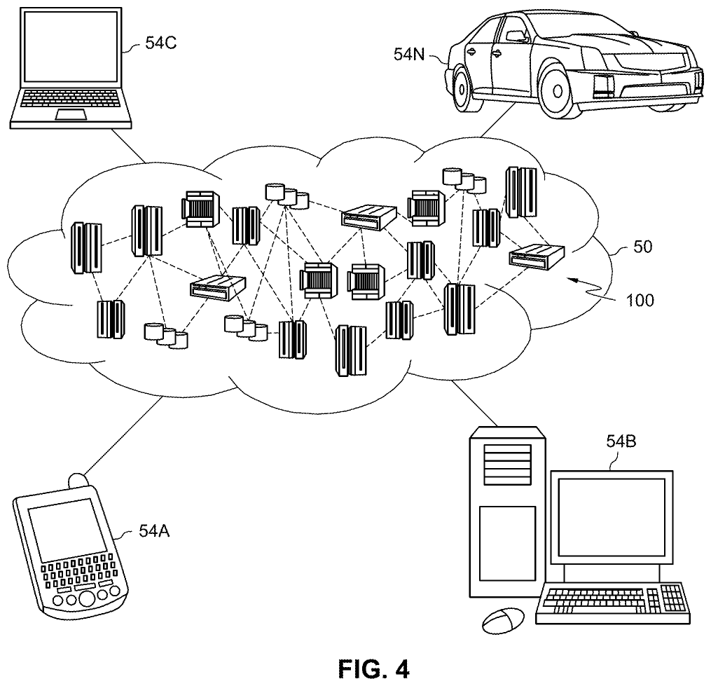

Referring now to FIG. 4, illustrative cloud computing environment 50 is depicted. As shown, cloud computing environment 50 comprises one or more cloud computing nodes 100 with which local computing devices used by cloud consumers, such as, for example, personal digital assistant (PDA) or cellular telephone 54A, desktop computer 54B, laptop computer 54C, and/or automobile computer system 54N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 100 and cloud computing environment 50 can communicate with any type of computerized device over any type of network and/or network addressable connection (e.g., using a web browser).

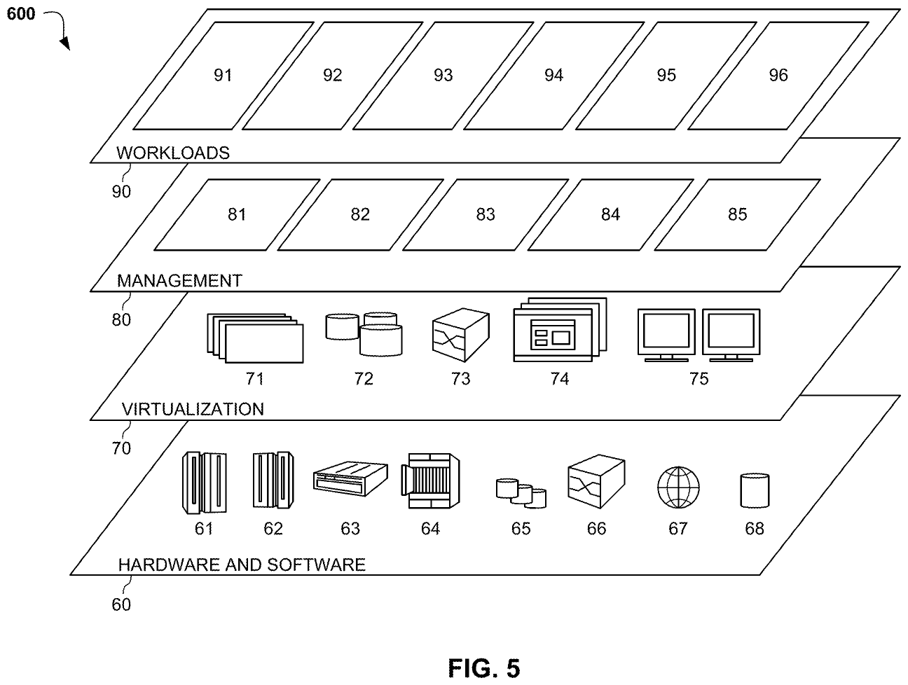

Referring now to FIG. 5, a set of functional abstraction layers 500 provided by cloud computing environment 50 is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and cognitive notifications choreographer 96. Cognitive notifications choreographer 96 may relate to analyzing data from smart sensors to generate a model of the user that predicts user stress level and based on the context of the predicted task and relevancy of the communication to the task and stress level of the user, dynamically reassign the communication to reduce the stress level of the user.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method for cognitive notification choreography, the method comprising:
receiving one or more communications and data from a sensor;
determining an activity of a user based on the received data, wherein the activity is determined by a trained neural network;
calculating a stress coefficient based on the activity and the data;
generating a cognitive notifications choreographer model from the stress coefficient and one or more communications; and
causing a communication software to reply to the one or more communications based on the cognitive notifications choreographer model determining the stress coefficient is above a threshold value.

2. The method of claim 1, wherein the sensor is a microphone, and wherein the data is a converted voice of the user to text.

3. The method of claim 1, wherein the stress coefficient derived from the activity and comprises a complexity, an importance of a task, a sentiment coefficient and a participation coefficient.

4. The method of claim 1, wherein the cognitive notifications choreographer model evaluates a stress factor and a relevancy of the one or more communications to a task.

5. The method of claim 1, wherein the sensor is a camera and wherein the data comprises a body language of the user to text.

6. The method of claim 1, further comprising:
determining whether each of the one or more communications is associated with the activity using topic modelling and corpus linguistic techniques.

7. The method of claim 1, further comprising:
displaying a dashboard comprising the one or more communications that are handled automatically.

8. A computer system for cognitive notification choreography, the computer system comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
receiving one or more communications and data from a sensor;
determining an activity of a user based on the received data, wherein the activity is determined by a trained neural network;
calculating a stress coefficient based on the activity and the data;
generating a cognitive notifications choreographer model from the stress coefficient and one or more communications; and
causing a communication software to reply to the one or more communications based on the cognitive notifications choreographer model determining the stress coefficient is above a threshold value.

9. The computer system of claim 8, wherein the sensor is a microphone, and wherein the data is a converted voice of the user to text.

10. The computer system of claim 8, wherein the stress coefficient derived from the activity and comprises a complexity, an importance of a task, a sentiment coefficient and a participation coefficient.

11. The computer system of claim 8, wherein the cognitive notifications choreographer model evaluates a stress factor and a relevancy of the one or more communications to a task.

12. The computer system of claim 8, wherein the sensor is a camera and wherein the data comprises a body language of the user to text.

13. The computer system of claim 8, further comprising:
determining whether each of the one or more communications is associated to the activity using topic modelling and corpus linguistic techniques.

14. The computer system of claim 8, further comprising:
displaying a dashboard comprising the one or more communications that are handled automatically.

15. A computer program product for cognitive notification choreography, the computer program product comprising:
one or more computer-readable tangible storage medium and program instructions stored on at least one of the one or more tangible storage medium, the program instructions executable by a processor, the program instructions comprising:
program instructions to receive one or more communications and data from a sensor;
program instructions to determine an activity of a user based on the received data, wherein the activity is determined by a trained neural network;
program instructions to calculate a stress coefficient based on the activity and the data;
program instructions to generate a cognitive notifications choreographer model from the stress coefficient and one or more communications; and
program instructions to cause a communication software to reply to the one or more communications based on the cognitive notifications choreographer model determining the stress coefficient is above a threshold value.

16. The computer program product of claim 15, wherein the sensor is a microphone, and wherein the data is a converted voice of the user to text.

17. The computer program product of claim 15, wherein the stress coefficient derived from the activity and comprises a complexity, an importance of a task, a sentiment coefficient and a participation coefficient.

18. The computer program product of claim 15, wherein the cognitive notifications choreographer model evaluates a stress factor and a relevancy of the one or more communications to a task.

19. The computer program product of claim 15, wherein the sensor is a camera and wherein the data comprises a body language of the user to text.

20. The computer program product of claim 15, further comprising:
program instructions to determine whether each of the one or more communications is associated to the activity using topic modelling and corpus linguistic techniques.
